(19) United States
(12) Patent Application Publication
(10) Pub. No.: US 2010/0235929 A1
(43) Pub. Date: Sep. 16, 2010

(54) Compositions and Methods for Producing Antibodies Having Human Idiotypes in Transgenic Birds

(75) Inventor: Roland Buelow, Palo Alto, CA (US)

Correspondence Address: ARNOLD & PORTER LLP;ATTN: IP DOCKETING DEPT., 555 TWELFTH STREET, N.W., WASHINGTON, DC, 20004-1206, US

(73) Assignee: Buelow Research Enterprises LLC, Palo Alto, CA (US)

(21) Appl. No.: 12/602,480

(22) PCT Filed: May 30, 2008
(86) PCT No.: PCT/US08/65424

§ 371 Date: May 28, 2010

Related U.S. Application Data
(60) Provisional application No. 60/941,615, filed on Jun. 1, 2007.

(51) Int. Cl. C12P 21/00; A01K 67/027; C07K 16/00; A61K 39/395; A61P 37/00; C12N 15/00

(52) U.S. Cl. 800/4; 800/19; 530/387.1; 530/388.1; 530/387.3; 424/130.1; 424/141.1; 800/21

(57) ABSTRACT

The invention relates to compositions and methods for producing antibodies having human idiotypes in non-human animals.

### CROSS-REFERENCE TO RELATED APPLICATIONS

This application claims priority to U.S. provisional patent application Ser. No. 60/941,615 filed 1 Jun. 2007, which is incorporated herein in its entirety by reference.

### FIELD

The invention relates to antibodies having human idiotypes and methods of making the same in non-human animals.

### BACKGROUND OF THE INVENTION

Antibodies are an important class of pharmaceutical products that have been successfully used in the treatment of various human diseases and conditions, including infectious diseases, cancer, allergic diseases, and graft-versus-host disease, as well as in the prevention of transplant rejection.

One problem associated with the therapeutic application of non-human immunoglobulins is the potential immunogenicity of the same in human patients. In order to reduce the immunogenicity of such preparations, various strategies for the production of partially human (humanized) and fully human antibodies have been developed.

The ability to produce transgenic antibodies having a human idiotype in non-human animals is particularly desirable as antigen binding determinants lie within the idiotype region, and non-human idiotypes are thought to contribute to the immunogenicity of current antibody therapeutics. Human idiotype is an especially important consideration in respect of monoclonal antibody therapeutics, which consist of a single idiotype delivered at relatively high concentration as opposed to the variety of idiotypes delivered at lower concentrations by a polyclonal antibody mixture. However, the production of human idiotype antibodies in birds has proven difficult due to gene conversion.

In animals such as human and mouse, there are multiple copies of V, D and J gene segments on the heavy chain locus, and multiple copies of V and J gene segments on the light chain locus. Antibody diversity in these animals is generated primarily by gene rearrangement, i.e., different combinations of gene segments form the rearranged heavy chain variable region and the rearranged light chain variable region. In birds, however, gene rearrangement does not play a significant role in the generation of antibody diversity. For example, in chicken, only one V gene segment (the one adjacent to the D region, or “the 3′ proximal V gene segment”), a D segment and one J segment are used in the heavy chain rearrangement; and only one V gene segment (the 3′ proximal V segment) and one J gene segment are used in the light chain rearrangement. Thus, in these animals, there is little diversity among initially rearranged variable region sequences resulting from junctional diversification.

In birds, such as chickens, further diversification of the rearranged Ig genes is achieved by gene conversion, a process in which short sequences derived from the upstream V gene segments replace short sequences within the V gene segment in the rearranged Ig gene. Accordingly, antibodies in gene converting animals display idiotypes that are encoded by fragments of more than one V gene segment. In contrast, idiotypes of human and rodent antibodies are always encoded by a single V gene segment.

Prior art methods of making humanized antibodies in birds have made use of artificial immunoglobulin loci comprising multiple V gene segments, providing for gene conversion and antibody gene diversification. Previously described recombinant loci have typically incorporated a single human V gene segment and multiple avian V gene segments upstream of the human V gene segment. While gene conversion increases antibody diversity in these birds, it also generates antibody idiotypes that differ from human antibody idiotypes, as birds with such loci generate V region sequences that are partially derived from chicken V gene sequences as well as the human sequence.

### SUMMARY OF THE INVENTION

The present invention stems from the finding that an artificial Ig locus comprising a single V gene segment, which is incapable of undergoing V gene conversion, is fully functional and readily rearranges in vivo to effect production of antibodies generated from a single V gene segment in transgenic birds. Further, a single human V gene segment, one or more human J segments, and one or more human or avian D gene segments in the case of a heavy chain locus, may be combined in such an artificial Ig locus to generate functional loci that provide for the generation of antibodies having a human idiotype in transgenic birds. Further, hypermutation at such artificial Ig loci increases antibody diversity and affinity range in a manner somewhat analogous to affinity maturation in humans as it is carried out on a sequence encoding a human idiotype maintained by the single human V gene segments within loci. Antibodies derived from such transgenic birds have a human idiotype, a feature highly desirable in antibody therapeutics.

Additionally, the inventor has found that, fortuitously, vectors comprising a single V gene segment are much easier to derive and much more stable than those comprising multiple V gene segments, providing for greater ease and efficiency of transgenic bird production.

Accordingly, in one aspect, the invention provides methods of making transgenic birds capable of producing immunoglobulins having human idiotypes. In one embodiment, the transgenic birds are capable of producing humanized immunoglobulins. In another embodiment, the transgenic birds are capable of producing fully human immunoglobulins. The methods comprise introducing artificial heavy and/or light chain loci of the invention into the genome of a bird, wherein the artificial loci each comprise a single V gene segment, which single V gene segment is a human V gene segment, wherein the artificial Ig loci are functional but incapable of V gene conversion.

In one embodiment, the methods comprise random integration of an artificial Ig locus of the invention.

In one embodiment, the methods comprise targeted integration of an artificial Ig locus of the invention.

In one embodiment, targeted integration involves the use of a meganuclease.

In one embodiment, targeted integration is done using homologous recombination.

In one embodiment, the methods involve inactivating one or more endogenous Ig loci of the bird genome. In one embodiment, the methods involve inactivating an endogenous Ig light chain locus. In one embodiment, the methods involve inactivating an endogenous Ig heavy chain locus. In one embodiment, the methods involve inactivating an endogenous Ig light chain locus and an endogenous Ig heavy chain locus.

In one embodiment, such inactivation is done using a meganuclease.

In one embodiment, such inactivation is done using homologous recombination.

In one embodiment, the transgenic bird is selected from the group consisting of chicken turkey, goose, duck, quail, and pheasant.

In one aspect, the invention provides transgenic birds produced by methods of the invention.

In one embodiment, the invention provides transgenic birds comprising a functional artificial Ig locus incapable of Ig gene conversion.

In one embodiment, the invention provides transgenic birds comprising a functional artificial Ig locus, which locus comprises a single V gene segment, which single V gene segment is a human V gene segment. In one embodiment, the artificial Ig locus is an Ig heavy chain locus. In one embodiment, the artificial Ig locus is an Ig light chain locus.

In a preferred embodiment, the transgenic birds lack a functional endogenous Ig light chain locus and/or a functional Ig heavy chain locus and are, accordingly, incapable of producing endogenous immunoglobulins.

In one embodiment, the invention provides transgenic birds comprising at least one artificial heavy chain Ig locus of the invention and at least one artificial light chain Ig locus of the invention, which birds are capable of producing antibodies having a human idiotype. In a preferred embodiment, the birds have an inactivated endogenous heavy chain Ig locus, an inactivated endogenous light chain locus, or both.

In one embodiment, the invention provides transgenic birds comprising an artificial Ig heavy chain locus and lacking a functional Ig light chain locus. Such transgenic birds are capable of producing heavy chain-only antibodies.

In one embodiment, the invention provides transgenic birds comprising two or more artificial heavy chain Ig loci of the invention in addition to at least one artificial light chain Ig locus of the invention. In a preferred embodiment, the two or more artificial heavy chain Ig loci of a transgenic bird comprise different human V gene segments.

In another embodiment, the invention provides transgenic birds comprising two or more artificial light chain Ig loci of the invention in addition to at least one artificial heavy chain Ig locus of the invention. In a preferred embodiment, the two or more artificial light chain Ig loci of a transgenic bird comprise different human V gene segments.

In one embodiment, the invention provides transgenic birds capable of producing chimeric antibodies having a human idiotype and bird-derived Fc-regions.

In one embodiment, the invention provides transgenic birds capable of producing antibodies having a human idiotype and human-derived Fc regions.

In one aspect, the invention provides vectors useful for producing transgenic birds of the invention. Such vectors comprise artificial Ig loci of the invention.

In one aspect, the invention provides artificial Ig loci. An artificial Ig locus of the invention comprises a single V gene segment, which single V gene segment is a human V gene segment.

In a preferred embodiment, the V gene segment encodes a germline or hypermutated human V-region amino acid sequence.

In one embodiment, an artificial Ig locus is a heavy chain locus. In a preferred embodiment, such an artificial Ig locus comprises one or more human J gene segments, one or more D gene segments, and one or more constant region genes. In a preferred embodiment, one or more D gene segments are human D gene segments. In a preferred embodiment, one or more constant region genes are human constant region genes.

In another embodiment, an artificial Ig locus is a light chain locus. In a preferred embodiment, such an artificial Ig locus comprises one or more human J gene segments and one or more constant region genes. In a preferred embodiment, one or more constant region genes are human constant region genes.

In a preferred embodiment, an artificial Ig locus of the invention is unrearranged.

In another embodiment, an artificial Ig locus of the invention is partially rearranged.

In another embodiment, an artificial Ig locus of the invention is fully rearranged.

In one aspect, the invention provides methods of making a vector comprising an artificial Ig locus of the invention.

In one aspect, the invention provides compositions and methods for making antibodies having human idiotypes in transgenic birds. The methods comprise immunizing a transgenic bird of the invention that is capable of making antibodies having human idiotype with an immunogen to generate antibodies having a human idiotype, which antibodies bind specifically to the immunogen.

In one embodiment, the invention provides methods for making antibodies with human idiotypes in a transgenic bird capable of making antibodies having human idiotypes. For the generation of polyclonal and monoclonal antibodies, preferably a plurality of such transgenic birds is immunized with an immunogen and birds producing antibodies specific for the immunogen are identified and used for the preparation of polyclonal antisera. Alternatively, birds producing antibodies specific for the immunogen can be used to generate monoclonal antibodies specific for the immunogen.

In a preferred embodiment, the method comprises immunizing a transgenic bird of the invention with an immunogen, wherein the transgenic bird has at least one artificial heavy chain Ig locus and at least one artificial light chain Ig locus, wherein each artificial Ig locus is functional and comprises multiple Ig gene segments, including a single human variable (V) gene segment, multiple J gene segments, one or more D gene segments in the case of heavy chain locus, and one or more constant region gene segments, wherein each of the single V-gene segments is functional and capable of being diversified by hypermutation but not gene conversion, wherein each of the single V-gene segments encodes a germline or hypermutated human V-region amino acid sequence. The transgenic bird is preferably incapable of producing endogenous immunoglobulin. In a preferred embodiment, the transgenic bird lacks a functional endogenous Ig light chain and/or a functional endogenous Ig heavy chain locus.

In one embodiment, the methods comprise immunizing a transgenic bird of the invention with an immunogen, wherein the transgenic bird comprises two or more artificial heavy chain Ig loci, wherein two or more of the artificial heavy chain Ig loci comprise different single human V gene segments.

In another embodiment, the methods comprise immunizing two or more transgenic birds of the invention with an immunogen, wherein two or more of the transgenic birds comprise artificial heavy chain Ig loci, which loci comprise different single human V gene segments.

In one embodiment, the methods comprise immunizing a transgenic bird of the invention with an immunogen, wherein the transgenic bird comprises two or more artificial light chain Ig loci, wherein two or more of the artificial light chain Ig loci comprise different single human V gene segments.

In another embodiment, the methods comprise immunizing two or more transgenic birds of the invention with an immunogen, wherein two or more of the transgenic birds comprise artificial light chain Ig loci, which loci comprise different single human V gene segments.

In one embodiment, the invention provides methods of producing heavy chain-only antibodies. The methods comprise immunizing a transgenic bird of the invention with an immunogen, wherein the transgenic bird comprises an artificial Ig heavy chain locus and lacks a functional Ig light chain locus.

In one aspect, the invention provides polyclonal antibodies derived from transgenic birds of the invention.

In one embodiment, the antibodies are chimeric antibodies having a human idiotype.

In one embodiment, the antibodies have a human idiotype and a human Fc region.

In one embodiment, the antibodies are heavy chain-only antibodies.

In one aspect, the invention provides monoclonal antibodies derived from transgenic birds of the invention.

In one embodiment, the monoclonal antibodies are chimeric antibodies having a human idiotype.

In a preferred embodiment, the monoclonal antibodies have a human idiotype and a human Fc region.

In one embodiment, the monoclonal antibodies are heavy chain-only antibodies.

In one aspect, the invention provides cells derived from transgenic birds of the invention.

In a preferred embodiment, the invention provides cells derived from the spleen of transgenic birds of the invention.

In a preferred embodiment, the invention provides B cells derived from transgenic birds of the invention, which B cells are capable of producing antibodies having a human idiotype.

In one aspect, the invention provides methods for making hybridomas capable of producing antibodies having a human idiotype. The methods comprise the use of cells derived from transgenic birds of the invention.

In one aspect, the invention provides hybridomas so produced.

In one aspect, the invention provides antibodies having a human idiotype, which antibodies are produced by a hybridoma of the invention.

In one aspect, the invention provides methods for making fully human monoclonal antibodies by recombinant means, comprising the use of rearranged antibody gene segments derived from transgenic birds of the invention, which rearranged antibody gene segments encode antibodies having a human idiotype.

In one aspect, the invention provides fully human monoclonal antibodies so produced.

In one aspect, the invention provides nucleic acids encoding monoclonal antibodies, which are isolated from cells that express a monoclonal antibody of the invention.

In one aspect, the invention provides nucleic acids encoding fully human monoclonal antibodies of the invention.

In one aspect, the invention provides methods for neutralizing or modulating the activity of an antigenic entity in a human body component. In one embodiment, the methods comprise contacting the body component with a polyclonal antisera composition of the invention, wherein the polyclonal antisera composition comprises immunoglobulin molecules that specifically bind to and neutralize or modulate the activity of the antigenic entity.

In one embodiment, the methods comprise contacting the body component with a monoclonal antibody of the invention, wherein the monoclonal antibody specifically binds to and neutralizes or modulates the activity of the antigenic entity.

In a preferred embodiment, the monoclonal antibody is a fully human monoclonal antibody.

In one embodiment, the antigenic entity is from an organism that causes an infectious disease.

In one embodiment, the antigenic entity is a cell surface molecule.

In one embodiment, the antigenic entity is a human cytokine or a human chemokine.

In one embodiment, the antigenic entity is a cell surface molecule on a malignant cancer cell.

In one aspect, the invention provides pharmaceutical compositions comprising an antibody of the invention, which antibody has a human idiotype.

In one aspect, the invention provides methods of treating a patient in need of treatment, comprising administering a therapeutically effective amount of a pharmaceutical composition of the invention to the patient.

### BRIEF DESCRIPTION OF THE DRAWINGS

FIG. 1. Artificial Immunoglobulin Heavy Chain Locus comprising a single human VH, human D elements, a single human JH and a chicken constant region containing Cμ, Cα and Cγ. Spacers between VH, D and JH are human, all other sequences are derived from chicken.

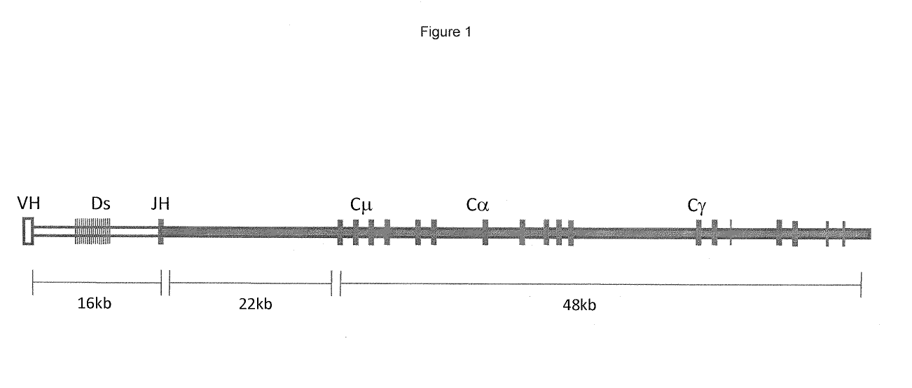

### DETAILED DESCRIPTION

By “artificial immunoglobulin locus” is meant an immunoglobulin locus comprising fragments of human and non-human immunoglobulin loci, including multiple immunoglobulin gene segments, which include a single human V-gene segment, multiple human J-gene segments, one or more D gene segments in the case of a heavy chain locus, and one or more constant region genes, wherein the human V gene encodes a germline or hypermutated human V-region amino acid sequences and wherein the artificial immunoglobulin locus is functional and capable of producing a repertoire of immunoglobulins diversified by hypermutation but not gene conversion, and wherein the rearranged artificial immunoglobulin locus encodes a heavy or light chain with a human idiotype. Artificial Ig locus as used herein can refer to unrearranged loci, partially rearranged loci, and rearranged loci. Artificial Ig loci include artificial Ig light chain loci and artificial Ig heavy chain loci. In one embodiment, an artificial Ig locus comprises a non-human C region gene and is capable of producing a repertoire of immunoglobulins including chimeric immunoglobulins having a non-human C region. In one embodiment, an artificial Ig locus comprises a human C region gene and is capable of producing a repertoire of immunoglobulins including immunoglobulins having a human C region. In one embodiment, an artificial Ig locus comprises an “artificial constant region gene”, by which is meant a constant region gene comprising nucleotide sequences derived from human and non-human constant regions genes.

In some embodiments, an artificial Ig heavy chain locus lacks CH1, or an equivalent sequence that allows the resultant immunoglobulin to circumvent the typical immunoglobulin: chaperone association. Such artificial loci provide for the production of heavy chain-only antibodies in transgenic birds which lack a functional Ig light chain locus and hence do not express functional Ig light chain. Such artificial Ig heavy chain loci are used in methods herein to produce transgenic birds lacking a functional Ig light chain locus, and comprising an artificial Ig heavy chain locus, which animals are capable of producing heavy chain-only antibodies. Alternatively, an artificial Ig locus may be manipulated in situ to disrupt CH1 or an equivalent region and generate an artificial Ig heavy chain locus that provides for the production of heavy chain-only antibodies. Regarding the production of heavy chain-only antibodies in light chain-deficient animals, see for example Zou et al., JEM, 204:3271-3283, 2007.

By “functional immunoglobulin locus” or “functional artificial immunoglobulin locus” is meant an Ig locus having the capacity to undergo gene rearrangement thereby producing a diversified repertoire of immunoglobulin molecules, or a locus that is so rearranged and capable of producing immunoglobulin molecules. Antibodies generated by a functional Ig locus are also referred to herein as “functional” antibodies.

By “human idiotype” is meant a polypeptide sequence present on a human antibody encoded by a immunoglobulin V-gene segment. The term “human idiotype” as used herein includes both naturally occurring sequences of a human antibody, as well as synthetic sequences substantially identical to the polypeptide found in naturally occurring human antibodies. By “substantially” is meant that the degree of amino acid sequence identity is at least about 85-95%. Preferably, the degree of amino acid sequence identity is greater than 90%, more preferably greater than 95%.

By “a chimeric antibody” or “a chimeric immunoglobulin” is meant an immunoglobulin molecule having at least a portion of a human immunoglobulin polypeptide sequence (or a polypeptide sequence encoded by a human Ig gene segment). The chimeric immunoglobulin molecules of the present invention are preferably immunoglobulins with bird-derived Fc-regions (Cμ or Cγ or Cε or Cα) and human idiotypes. Such immunoglobulins can be isolated from a transgenic bird engineered to produce chimeric immunoglobulin molecules.

By “artificial Fc-region” is meant an Fc-region encoded by an artificial constant region gene.

The term “Ig gene segment” as used herein refers to segments of DNA encoding various portions of an Ig molecule, which are present in the germline of non-human animals and humans, and which are brought together in B cells to form rearranged Ig genes. Thus, Ig gene segments as used herein include V gene segments, D gene segments, J gene segments and C region gene segments.

The term “human Ig gene segment” as used herein includes both naturally occurring sequences of a human Ig gene segment, degenerate forms of naturally occurring sequences of a human Ig gene segment, as well as synthetic sequences that encode a polypeptide sequence substantially identical to the polypeptide encoded by a naturally occurring sequence of a human Ig gene segment. By “substantially” is meant that the degree of amino acid sequence identity is at least about 85-95%. Preferably, the degree of amino acid sequence identity is greater than 90%, more preferably greater than 95%.

By “meganuclease” is meant an endodeoxyribonuclease that recognizes long recognition sites in DNA, preferably at least 12, more preferably at least 13, more preferably at least 14, more preferably at least 15, more preferably at least 16, more preferably at least 17, and most preferably at least 18 nucleotides in length. Meganucleases include zinc-finger nucleases, naturally occurring homing endonucleases and custom engineered zinc-finger nucleases and homing endonucleases. What is required for use in the invention is that the meganuclease recognize a meganuclease target sequence present in or proximal to an endogenous Ig locus in the subject animal such that a functional mutation may be introduced in the Ig locus by the action of the induced meganuclease. For more discussion of meganucleases, see, for example, U.S. Patent Application Publication Nos. 20060206949, 20060153826, 20040002092, 20060078552, and 20050064474. Zinc-finger nucleases with altered specificity can be generated by combining individual zinc fingers with different triplet targets. The specificity of naturally occurring homing endonucleases can be altered by structure-based protein engineering. For example, see Proteus and Carroll, nature biotechnology 23(8):967-97, 2005.

An animal having a “germline inactivated Ig locus”, or “germline inactivated endogenous Ig locus”, or “germline mutation in an endogenous Ig locus”, has an inactivated endogenous Ig locus in every cell, i.e., every somatic and germ cell. In the present invention, animals having germline inactivated loci are produced by mutation, as effected by the action of a meganuclease in a germ cell which gives rise to the resultant animal, or a predecessor thereof. In one embodiment, an entire endogenous Ig heavy chain and/or Ig light chain locus, or large parts thereof, is deleted from the genome of the subject animal. Such animals are also referred to as comprising an endogenous locus that has been inactivated.

Transgenic Birds that Produce Antibodies Having Human Idiotype, and Methods of Making the Same

In one aspect of the invention, methods of making transgenic birds capable of producing immunoglobulins with human idiotypes are provided. The methods involve the integration of one or more artificial heavy chain Ig loci and/or one or more artificial light chain Ig loci into the bird's genome. Where heavy chain-only antibodies are desired, only an artificial Ig heavy chain locus is used. Otherwise, heavy chain and light chain artificial loci are used. Regardless of the chromosomal location, an artificial Ig locus of the present invention is a functional immunoglobulin locus and has the capacity to undergo gene rearrangement thereby producing a diversified repertoire of immunoglobulin molecules.

In one embodiment, antibodies encoded by the artificial loci of the present invention have idiotypes encoded by a single heavy chain V-gene segment and a single light chain V gene segment. Rearranged V-genes in artificial loci of the present invention cannot be diversified by gene conversion because said loci contain single V gene segments.

According to the present invention, a transgenic bird capable of making immunoglobulins with human idiotypes is made by introducing into a recipient cell or cells of a bird one or more of the transgenic vectors which carry an artificial Ig locus, and deriving a bird from the genetically modified recipient cell or cells.

The transgenic vector containing an artificial Ig locus is introduced into the recipient cell or cells and then integrated into the genome of the recipient cell or cells by random integration or by targeted integration.

For random integration, a transgenic vector containing an artificial Ig locus can be introduced into an animal recipient cell by standard transgenic technology. For example, a transgenic vector can be directly injected into the pronucleus of a fertilized oocyte. A transgenic vector can also be introduced by co-incubation of sperm with the transgenic vector before fertilization of the oocyte. Transgenic animals can be developed from fertilized oocytes. Another way to introduce a transgenic vector is by transfecting embryonic stem cells, primordial germ cells or other pluripotent cells and subsequently injecting the genetically modified cells into developing embryos. Alternatively, a transgenic vector (naked or in combination with facilitating reagents) can be directly injected into a developing embryo. Ultimately, chimeric transgenic animals are produced from the embryos which contain the artificial Ig transgene integrated in the genome of at least some somatic cells of the transgenic animal. In another embodiment, the transgenic vector is introduced into the genome or a bird cell and an animal is derived from the transfected cell by nuclear transfer cloning.

For targeted integration, a transgenic vector can be introduced into appropriate animal recipient cells such as embryonic stem cells or already differentiated somatic cells. Afterwards, cells in which the transgene has integrated into the animal genome and has replaced the corresponding endogenous Ig locus by homologous recombination can be selected by standard methods. The selected cells may then be fused with enucleated nuclear transfer unit cells, e.g. oocytes or embryonic stem cells, cells which are totipotent and capable of forming a functional neonate. Fusion is performed in accordance with conventional techniques which are well established. See, for example, Cibelli et al., Science (1998) 280:1256. Enucleation of oocytes and nuclear transfer can also be performed by microsurgery using injection pipettes. (See, for example, Wakayama et al., Nature (1998) 394:369.) The resulting cells are then cultivated in an appropriate medium, and transferred into synchronized recipients for generating transgenic animals. Alternatively, the selected genetically modified cells can be injected into developing embryos which are subsequently developed into chimeric animals.

The frequency of targeted integration can be increased by double-strand DNA cleavage using site-specific meganucleases. For integration into endogenous immunoglobulin loci a site specific meganuclease may be used.

In one embodiment, the transgenic birds are nullizygous for Ig heavy chain and/or Ig light chain loci.

In one embodiment, a transgene containing an artificial Ig locus is integrated into the genome of recipient cells (such as fertilized oocyte or developing embryos) derived from a bird with an inactivated endogenous heavy and/or light chain locus and therefore incapable of expressing endogenous bird immunoglobulins. The use of such birds permits preferential expression of antibodies with human idiotypes from the artificial transgenic Ig locus. Alternatively, breeding with transgenic birds having inactivated endogenous Ig loci can be done to obtain birds nullizygous for endogenous Ig loci and comprising an artificial Ig locus.

Inactivation of endogenous bird immunoglobulin loci may be performed by using meganucleases specific for bird immunoglobulin sequences in or proximal to heavy and or light chain loci. In one embodiment double-strand breaks may be induced by injection of a meganuclease into fertilized oocytes. Alternatively, RNA or expression vectors encoding a meganuclease may be injected. In one embodiment, meganuclease is expressed in a germ cell, which may include, for example, a spermatogonial stem cell, to induce double-strand breaks. In a preferred embodiment of the present invention a vector encoding a site-specific meganuclease is integrated into the bird genome. Expression of the transgene encoding the meganuclease in germ cells will result in double-strand breaks in endogenous Ig loci and subsequent mutation of the restriction site. Mating of such transgenic animals results in offspring with mutated/inactivated immunoglobulin loci. To minimize cytotoxic effects associated with expression of a particular meganuclease it's expression may be controlled using inducible promoters, e.g., heat-inducible promoters, radiation-inducible promoters, tetracycline operon, hormone inducible promoters, and promoters inducible by dimerization of transactivators, and the like. For example, see Vilaboa et al., Current Gene Therapy, 6:421-438, 2006.

Alternatively, endogenous bird immunoglobulin loci may be inactivated using homologous recombination with our without meganucleases. In one embodiment, a transgene containing an artificial Ig locus is integrated into an endogenous Ig locus, thereby inactivating the endogenous Ig locus.

In one embodiment, the invention provides transgenic birds comprising an artificial Ig heavy chain locus and lacking a functional Ig light chain locus. Such transgenic birds find use in the production of heavy chain-only antibodies.

Inactivation of Endogenous Ig Loci

Inactivation of endogenous Ig loci is done using meganucleases specific for immunoglobulin gene fragments in heavy or light chain loci endogenous to the subject animal. In one embodiment double-strand breaks may be induced by injection of a meganuclease into germ cells, fertilized oocytes or embryos. Alternatively, expression vectors or nucleic acid encoding a meganuclease and capable of being expressed in germ cells, fertilized oocytes or embryos may be injected into the same.

In one embodiment, the method involves transfecting germ cells, which may include precursors thereof such as spermatagonial stem cells, in vitro or in vivo with a meganuclease encoding nucleic acid or expression construct. For example, see Ryu et al., J. Androl., 28:353-360, 2007; Orwig et al., Biol. Report, 67:874-879, 2002.

In a preferred embodiment, a meganuclease expression construct is integrated into the genome of the subject animal. Expression of the transgene encoding the meganuclease in germ cells will result in double-strand breaks in endogenous Ig loci and subsequent mutation of the restriction site. Mating of such transgenic animals results in offspring with mutated/inactivated immunoglobulin loci.

In a highly preferred embodiment of the present invention, a regulatable meganuclease expression construct is integrated into the genome of the subject animal, which regulatable construct is inducible in germ cells. Such constructs provide for minimization of cytotoxic effects associated with expression of a particular meganuclease through controlled expression via inducible promoters, e.g., heat-inducible promoters, radiation-inducible promoters, tetracycline operon, hormone inducible promoters, and promoters inducible by dimerization of transactivators, and the like. For example, see Vilaboa et al., Current Gene Therapy, 6:421-438, 2006.

Alternatively, meganuclease expression may be induced in an embryo derived from the germ cell.

In one embodiment, a single meganuclease is expressed in a germ cell, wherein the meganuclease recognizes a target sequence in or proximal to an immunoglobulin locus endogenous to the germ cell of the subject animal. In a preferred embodiment, the meganuclease target sequence is in or proximal to a J gene segment. In another preferred embodiment, the meganuclease target sequence is in or proximal to an immunoglobulin constant region gene. In a preferred embodiment, the immunoglobulin constant region gene encodes immunoglobulin μ.

In a preferred embodiment, at least two meganucleases having distinct target sequences are used. The at least two meganucleases are expressed in a germ cell, wherein the meganucleases recognize distinct target sequences in or proximal to an immunoglobulin locus endogenous to the germ cell of the subject animal.

In a preferred embodiment, the first and second meganucleases target J gene segments. In one embodiment, the first and second meganuclease target sequences are, taken together, upstream and downstream of one or more J gene segments within the endogenous Ig locus, and cleavage by the first and second encoded meganucleases produces deletion of a genomic DNA segment comprising the one or more J gene segments.

In another embodiment, the first and second meganucleases target constant region gene segments. In one embodiment, the first and second meganuclease target sequences are, taken together, upstream and downstream of one or more immunoglobulin constant region gene segments, and cleavage by the first and second encoded meganucleases produces deletion of a genomic DNA segment comprising the one or more immunoglobulin constant region gene segments. In a preferred embodiment, the constant region gene encodes immunoglobulin μ.

In one embodiment, at least one meganuclease is used to disrupt the CH1 region of an endogenous Ig heavy chain locus, leaving the remainder of the locus intact and capable of producing an Ig heavy chain that circumvents the typical immunoglobulin:chaperone association. Preferably, this CH1 targeting is done in an animal lacking a functional Ig light chain locus. Such targeting in such animals is useful for producing heavy chain-only antibodies.

In one embodiment, more than one meganuclease is used to target CH1 within the Ig heavy chain locus.

In preferred embodiments, the breeding strategies used are designed to obtain animals that are nullizygous for endogenous Ig light chain and/or endogenous Ig heavy chain.

Artificial Immunoglobulin Loci, and Vectors Comprising the Same

The present invention is further directed to novel artificial Ig loci and their use in making transgenic birds which produce antibodies with human idiotypes. The artificial Ig loci contain constant region elements, one human V gene segment, one or more D gene segments in the case of a heavy chain locus, and one or more J gene segments. Regulatory elements like promoters, enhancers, switch regions, recombination signals, and the like may be of human or animal origin. In heavy chain loci human or non-human D gene segments are included in the artificial Ig loci.

In one embodiment, the present invention provides transgenic constructs containing an artificial Ig heavy chain locus which include regulatory elements, a V-region with one human V gene segment, a D-region with one or more D gene segments, a J-region with one or more human J gene segments, and a C-region with one or more bird and/or human constant region genes. The gene segments in such artificial heavy chain locus are juxtaposed with respect to each other in an unrearranged configuration (or “the germline configuration”), or in a partially or fully rearranged configuration. The unrearranged artificial heavy chain locus has the capacity to undergo gene rearrangement in the bird thereby producing a diversified repertoire of heavy chains having human idiotypes. The rearranged V gene segment in the artificial heavy chain locus of the present invention cannot be diversified by gene conversion because the artificial heavy chain locus contains a single human V gene.

A human VH segment encompasses naturally occurring sequences of a human VH gene segment, degenerate forms of naturally occurring sequences of a human VH gene segment, as well as synthetic sequences that encode a polypeptide sequence substantially (i.e., at least about 85%-95%) identical to a human heavy chain V domain polypeptide.

In another embodiment, the present invention provides transgenic constructs containing an artificial light chain locus capable of undergoing gene rearrangement in the host animal thereby producing a diversified repertoire of light chains having human idiotypes. The rearranged V gene segment in the artificial heavy chain locus of the present invention cannot be diversified by gene conversion because the artificial light chain locus contains a single human V gene.

The humanized light locus includes regulatory elements, a V-region with a single human V gene segment, a J-region with one or more human J gene segments, and a C-region with one or more bird and/or human constant region gene segments. The gene segments in the humanized light chain locus are juxtaposed in an unrearranged configuration (or “the germline configuration”), or fully rearranged configuration.

A human VL segment encompasses naturally occurring sequences of a human VL gene segment, degenerate forms of naturally occurring sequences of a human VL gene segment, as well as synthetic sequences that encode a polypeptide sequence substantially (i.e., at least about 90%-95%) identical to a human light chain V domain polypeptide.

Another aspect of the present invention is directed to methods of making a transgenic vector containing an artificial Ig locus. Such methods involve isolating Ig loci or fragments thereof, and combining it with one or several DNA fragments comprising sequences encoding human V region elements. The Ig gene segment(s) are inserted into the artificial Ig locus or a portion thereof by ligation or homologous recombination in such a way as to retain the capacity of the locus of undergoing effective gene rearrangement in the bird.

Preferably, an Ig locus from a bird is isolated by screening a library of plasmids, cosmids, YACs or BACs, and the like, prepared from the genomic DNA of the rat. YAC clones can carry DNA fragments of up to 2 megabases, thus an entire animal heavy chain locus or a large portion thereof can be isolated in one YAC clone, or reconstructed to be contained in one YAC clone. BAC clones are capable of carrying DNA fragments of smaller sizes (about 150-250 kb). However, multiple BAC clones containing overlapping fragments of an Ig locus can be separately altered and subsequently injected together into an animal recipient cell, wherein the overlapping fragments recombine in the recipient animal cell to generate a continuous Ig locus.

Human Ig gene segments can be integrated into the Ig locus on a vector (e.g., a BAC clone) by a variety of methods, including ligation of DNA fragments, or insertion of DNA fragments by homologous recombination. Integration of the human Ig gene segments is done in such a way that the human Ig gene segment is operably linked to the host animal sequence in the transgene to produce a functional artificial Ig locus. Homologous recombination can be performed in bacteria, yeast and other cells with a high frequency of homologous recombination events. Engineered YACs and BACs can be readily isolated from the cells and used in making transgenic animals.

Preparation of Antibodies Having Human Idiotype

In another embodiment of the present invention, a preparation of antibodies having a human idiotype is provided.

While antibodies may be prepared from transgenic birds comprising an artificial light chain Ig locus but lacking an artificial heavy chain locus, as well as from transgenic birds comprising an artificial heavy chain locus but lacking an artificial light chain locus, these are less preferred embodiments that yield antibodies lacking a complete human idiotype. What is preferred is the use of transgenic birds comprising at least one artificial heavy chain locus and at least one artificial light chain locus, which yield antibodies having human heavy chain and human light chain idiotypes.

By “a preparation of antibodies having a human idiotype” is meant an isolated antibody product or a purified antibody product prepared from a transgenic bird of the invention (e.g., serum or egg yolk of the animal) or from cells derived from a transgenic bird of the invention (e.g., a B-cell or a hybridoma cell).

An antibody preparation can be a preparation of polyclonal or monoclonal antibodies.

Once transgenic birds capable of producing a diversified repertoire of antibodies with human idiotypes have been made, antibody preparations against an immunogen can be readily obtained by immunizing the animals with the immunogen. For the production of antibodies a group of birds with different artificial immunoglobulin loci are immunized. The artificial immunoglobulin loci in these birds have different V gene segments. Therefore, immunization of a large group of birds with different artificial immunoglobulin loci allows antibody production using a large number of different V gene segments. Alternatively, a number of different artificial immunoglobulin loci may be combined in a single bird by breeding and thereby the number of animals with different artificial loci required for immunization can be reduced. Following immunization, animals producing antibody specific for the immunogen are identified an antisera can be pooled.

In one embodiment, the invention provides heavy chain-only antibodies that are obtained from a transgenic bird of the invention, which transgenic bird comprises an artificial Ig heavy chain locus and lacks a functional Ig light chain locus.

A variety of antigens can be used to immunize transgenic birds. Such antigens include, microorganism, e.g. viruses and unicellular organisms (such as bacteria and fungi), alive, attenuated or dead, fragments of the microorganisms, or antigenic molecules isolated from the microorganisms.

Preferred bacterial antigens for use in immunizing an animal include purified antigens from Staphylococcus aureus such as capsular polysaccharides type 5 and 8, recombinant versions of virulence factors such as alpha-toxin, adhesin binding proteins, collagen binding proteins, and fibronectin binding proteins. Preferred bacterial antigens also include an attenuated version of S. aureus, Pseudomonas aeruginosa, enterococcus, enterobacter, and Klebsiella pneumoniae, or culture supernatant from these bacteria cells. Other bacterial antigens which can be used in immunization include purified lipopolysaccharide (LPS), capsular antigens, capsular polysaccharides and/or recombinant versions of the outer membrane proteins, fibronectin binding proteins, endotoxin, and exotoxin from Pseudomonas aeruginosa, enterococcus, enterobacter, and Klebsiella pneumoniae.

Preferred antigens for the generation of antibodies against fungi include attenuated version of fungi or outer membrane proteins thereof, which fungi include, but are not limited to, Candida albicans, Candida parapsilosis, Candida tropicalis, and Cryptococcus neoformans.

Preferred antigens for use in immunization in order to generate antibodies against viruses include the envelop proteins and attenuated versions of viruses which include, but are not limited to respiratory synctial virus (RSV) (particularly the F-Protein), Hepatitis C virus (HCV), Hepatitis B virus (HBV), cytomegalovirus (CMV), EBV, and HSV.

Antibodies specific for cancer can be generated by immunizing transgenic rats with isolated tumor cells or tumor cell lines; tumor-associated antigens which include, but are not limited to, Her-2-neu antigen (antibodies against which are useful for the treatment of breast cancer); CD20, CD22 and CD53 antigens (antibodies against which are useful for the treatment of B cell lymphomas), (3) prostate specific membrane antigen (PMSA) (antibodies against which are useful for the treatment of prostate cancer), and 17-1A molecule (antibodies against which are useful for the treatment of colon cancer).

The antigens can be administered to a transgenic bird in any convenient manner, with or without an adjuvant, and can be administered in accordance with a predetermined schedule.

After immunization, serum or milk from the immunized transgenic animals can be fractionated for the purification of pharmaceutical grade polyclonal antibodies specific for the antigen. Antibodies can also be made by fractionating egg yolks. A concentrated, purified immunoglobulin fraction may be obtained by chromatography (affinity, ionic exchange, gel filtration, etc.), selective precipitation with salts such as ammonium sulfate, organic solvents such as ethanol, or polymers such as polyethyleneglycol.

For making a monoclonal antibody, spleen cells are isolated from the immunized transgenic animal and used either in cell fusion with transformed cell lines for the production of hybridomas, or cDNAs encoding antibodies are cloned by standard molecular biology techniques and expressed in transfected cells. The procedures for making monoclonal antibodies are well established in the art. See, e.g., WO 97/16537 (“Stable Chicken B-cell Line And Method of Use Thereof”), and EP 0491 057 B1 (“Hybridoma Which Produces Avian Specific Immunoglobulin G”), the disclosures of which are incorporated herein by reference. In vitro production of monoclonal antibodies from cloned cDNA molecules has been described by Andris-Widhopf et al., “Methods for the generation of chicken monoclonal antibody fragments by phage display”, Immunol Methods 242: 159 (2000), and by Burton, D. R., “Phage display”, Immunotechnology 1: 87 (1995), the disclosures of which are incorporated herein by reference.

Once chimeric monoclonal antibodies with human idiotypes have been generated, such chimeric antibodies can be easily converted into fully human antibodies using standard molecular biology techniques. Fully human monoclonal antibodies are not immunogenic in humans and are appropriate for use in the therapeutic treatment of human subjects.

Antibodies of the Invention Include Heavy Chain-Only Antibodies

In one embodiment, transgenic animals which lack a functional Ig light chain locus, and comprising an artificial heavy chain locus, are immunized with antigen to produce heavy chain-only antibodies that specifically bind to antigen.

In one embodiment, the invention provides monoclonal antibody producing cells derived from such animals, as well as nucleic acids derived therefrom. Also provided are hybridomas derived therefrom. Also provided are fully human heavy chain-only antibodies, as well as encoding nucleic acids, derived therefrom.

Teachings on heavy chain-only antibodies are found in the art. For example, see PCT publications WO02085944, WO02085945, WO2006008548, and WO2007096779. See also U.S. Pat. No. 5,840,526; U.S. Pat. No. 5,874,541; U.S. Pat. No. 6,005,079; U.S. Pat. No. 6,765,087; U.S. Pat. No. 5,800,988; EP 1589107; WO 9734103; and U.S. Pat. No. 6,015,695.

Pharmaceutical Compositions

In a further embodiment of the present invention, purified monoclonal or polyclonal antibodies are admixed with an appropriate pharmaceutical carrier suitable for administration in primates especially humans, to provide pharmaceutical compositions.

Pharmaceutically acceptable carriers which can be employed in the present pharmaceutical compositions can be any and all solvents, dispersion media, isotonic agents and the like. Except insofar as any conventional media, agent, diluent or carrier is detrimental to the recipient or to the therapeutic effectiveness of the antibodies contained therein, its use in the pharmaceutical compositions of the present invention is appropriate.

The carrier can be liquid, semi-solid, e.g. pastes, or solid carriers. Examples of carriers include oils, water, saline solutions, alcohol, sugar, gel, lipids, liposomes, resins, porous matrices, binders, fillers, coatings, preservatives and the like, or combinations thereof.

Transgenic animals produced by any of the foregoing methods form another embodiment of the present invention. The transgenic animals have at least one, i.e., one or more, artificial Ig loci in the genome, from which a functional repertoire of antibodies with human idiotypes is produced.

In another preferred embodiment, the present invention provides transgenic birds having one or more artificial Ig loci in the genome. The transgenic birds of the present invention are capable of rearranging the artificial Ig loci, and expressing a functional repertoire of antibodies with human idiotypes.

Cells derived from the transgenic animals of the present invention, such as B cells or cell lines established from a transgenic animal immunized against an antigen, are also part of the present invention.

Methods of Treatment

In a further aspect of the present invention, methods are provided for treating a disease in a vertebrate, preferably a mammal, preferably a primate, with human subjects being an especially preferred embodiment, by administering a purified antibody composition of the invention desirable for treating such disease.

The antibody compositions can be used to bind and neutralize or modulate an antigenic entity in human body tissues that causes or contributes to disease or that elicits undesired or abnormal immune responses. An “antigenic entity” is herein defined to encompass any soluble or cell surface bound molecules including proteins, as well as cells or infectious disease-causing organisms or agents that are at least capable of binding to an antibody and preferably are also capable of stimulating an immune response.

Administration of an antibody composition against an infectious agent as a monotherapy or in combination with chemotherapy results in elimination of infectious particles. A single administration of antibodies decreases the number of infectious particles generally 10 to 100 fold, more commonly more than 1000-fold. Similarly, antibody therapy in patients with a malignant disease employed as a monotherapy or in combination with chemotherapy reduces the number of malignant cells generally 10 to 100 fold, or more than 1000-fold. Therapy may be repeated over an extended amount of time to assure the complete elimination of infectious particles, malignant cells, etc. In some instances, therapy with antibody preparations will be continued for extended periods of time in the absence of detectable amounts of infectious particles or undesirable cells.

Similarly, the use of antibody therapy for the modulation of immune responses may consist of single or multiple administrations of therapeutic antibodies. Therapy may be continued for extended periods of time in the absence of any disease symptoms.

The subject treatment may be employed in conjunction with chemotherapy at dosages sufficient to inhibit infectious disease or malignancies. In autoimmune disease patients or transplant recipients, antibody therapy may be employed in conjunction with immunosuppressive therapy at dosages sufficient to inhibit immune reactions.

### EXPERIMENTAL

### Example 1

### Generation of Transgenic Chickens Expressing Antibodies with Human Idiotypes

Primordial germ cells (PGCs) are isolated and cultured as described by van de Lavoir et al. (Nature 441: 766-769, 2006). Blood is taken from the vasculature of stage 14-17 (H&H) embryos and cultured in 96-well plates with mitotically inactivated STO cells. Cells are grown in KO-DMEM (Invitrogen) that was conditioned with BRL cells. The medium contains 7.5% FCS, 2.5% chicken serum, 2 mM glutamine, 1 mM pyruvate, 1× nucleosides, 1× non-essential amino acids and 0.1 mM β-mercaptoethanol, 6 ng/ml SCF and 4 ng/ml human recombinant FGF.

PGCs are transfected with an artificial heavy chain immunoglobulin locus comprising a single human VH element, a chicken D-region, a human JH region, a chicken intron enhancer, and chicken Cμ and Cγ, and a selection marker gene flanked by loxP sites. For transfection PGCs are resuspended in electroporation buffer (Speciality Media). Following the addition of linearized DNA one exponential decay pulse (200V, with 900-100 μF) is given. Transfected cells are grown for several days and transgenic cells are isolated. Selection of transgenic cells is accomplished using antibiotic selection (for example, neomycin or puromycin).

Subsequently, genetically modified cells are injected using a 37 μm diameter needle into the anterior portion of the sinus terminales of a stage 13-15 (H&H) embryo. The injected embryos are transferred to a second surrogate shell for incubation until hatching.

Somatic chimerism of hatched chickens is evaluated by PCR. Germ-line chimerism is assessed by mating of somatic chimeras. The rate of germline transmission ranges from <1 to 80%. Transgenic chicken expressing artificial immunoglobulin heavy chains with human idiotypes are mated with chicken expressing artificial immunoglobulin light chains with human idiotypes. Offsprings are screened for expression of immunoglobulin molecules containing human heavy and light chain elements.

All citations are expressly incorporated herein in their entirety by reference.

We claim:

1. A transgenic bird comprising at least one artificial Ig locus, wherein said artificial Ig locus comprises multiple Ig gene segments, including a single human variable (V) gene segment, one or more J gene segments, and one or more constant region gene segments, wherein said artificial Ig locus is functional, wherein said single V-gene segment is functional and capable of being diversified by hypermutation but not gene conversion, and wherein said single V-gene segment encodes a germline or hypermutated human V-region amino acid sequence.

2. The transgenic bird according to claim 1, wherein said artificial Ig locus is an artificial Ig heavy chain locus.

3. The transgenic bird according to claim 1, wherein said artificial Ig locus is an artificial Ig light chain locus.

4. The transgenic bird according to claim 1, further comprising a second artificial Ig locus, which second artificial Ig locus comprises multiple Ig gene segments, including a single human variable (V) gene segment, one or more J gene segments, and one or more constant region gene segments, wherein said second artificial Ig locus is functional, wherein said single V-gene segment is functional and capable of being diversified by hypermutation but not gene conversion, and wherein said single V-gene segment encodes a germline or hypermutated human V-region amino acid sequence.

5. The transgenic bird according to claim 4, wherein the genome of said transgenic bird comprises a functional artificial Ig heavy chain locus and a functional artificial Ig light chain locus, and wherein said functional artificial Ig loci each comprise a single variable (V) gene segment which V gene segment is a germline or hypermutated human V gene segment.

6. The transgenic bird according to claim 1, wherein said transgenic bird is a chicken, turkey, quail, duck, pheasant or goose.

7. The transgenic bird according to claim 1, wherein said transgenic bird comprises an inactivated endogenous Ig locus.

8. The transgenic bird according to claim 2, wherein said transgenic bird lacks a functional Ig light chain locus.

9. A method for producing antibodies having a human idiotype, comprising immunizing a transgenic bird according to claim 5 with an immunogen.

10. The method according to claim 9, further comprising immunizing a second transgenic bird with said immunogen, wherein said second transgenic bird comprises a second functional artificial heavy chain Ig locus, and wherein said second functional artificial heavy chain Ig locus comprises a second single V gene segment which V gene segment is a germline or hypermutated human V gene segment that differs from the V gene segment in the functional artificial heavy chain Ig locus of the first transgenic bird.

11. The method according to claim 9, further comprising immunizing a second transgenic bird with said immunogen, wherein said second transgenic bird comprises a second functional artificial light chain Ig locus, and wherein said second functional artificial light chain Ig locus comprises a second single V gene segment which V gene segment is a germline or hypermutated human V gene segment that differs from the V gene segment in the functional artificial light chain Ig locus of the first transgenic bird.

12. A method for producing heavy chain-only antibodies, comprising immunizing a transgenic bird according to claim 8 with an immunogen.

13. The method according to claim 9, wherein said antibody is a monoclonal antibody.

14. The method according to claim 9, wherein said antibody is a humanized monoclonal antibody.

15. A polyclonal antiserum produced by the method according to claim 9.

16. A monoclonal antibody produced by the method according to claim 13.

17. A humanized monoclonal antibody produced by the method according to claim 14.

18. A heavy chain-only antibody produced by the method according to claim 12.

19. A method for neutralizing an antigenic entity in a human body component, said method comprising: contacting said body component with an antisera composition according to claim 15, whereby said immunoglobulin protein molecules in said antisera composition specifically bind and neutralize said antigenic entity.

20. A method for neutralizing an antigenic entity in a human body component, said method comprising: contacting said body component with the monoclonal antibody according to claim 16 or 17, whereby said monoclonal antibody specifically binds and neutralizes said antigenic entity.

21. A method for producing a transgenic bird according to claim 7, comprising expressing at least one meganuclease in a germ cell, fertilized oocyte or embryo, to generate a viable germ cell having at least one inactivated endogenous Ig locus wherein said meganuclease recognizes a meganuclease target sequence present in or proximal to said endogenous Ig locus, and deriving a transgenic bird from said viable germ cell having at least one inactivated endogenous Ig locus, or a germ cell descendant thereof.

22. The method according to claim 21, wherein said meganuclease target sequence is present in or proximal to a J gene segment within said at least one endogenous Ig locus.

23. The method according to claim 21, wherein said meganuclease target sequence is present in or proximal to an immunoglobulin constant region gene.

24. The method according to claim 21, further comprising expressing a second meganuclease in said germ cell, fertilized oocyte or embryo, wherein said second meganuclease recognizes a second meganuclease target sequence present in or proximal to said endogenous Ig locus.
